(19) United States
(12) Patent Application Publication
(10) Pub. No.: US 2022/0115095 A1
(43) Pub. Date: Apr. 14, 2022

(54) SYSTEM AND METHOD FOR MULTI CHIRAL DETECTION

(71) Applicant: TECHNION RESEARCH & DEVELOPMENT FOUNDATION LIMITED, Haifa (IL)

(72) Inventors: Oren COHEN, Haifa (IL); Ofer NEUFELD, Haifa (IL); Or PELEG, Adi (IL); Omri WENGROWICZ, Kfar-Sava (IL)

(21) Appl. No.: 17/501,261

(22) Filed: Oct. 14, 2021

Related U.S. Application Data
(60) Provisional application No. 63/091,515, filed on Oct. 14, 2020.

(51) Int. Cl. G16C 20/70; G16C 20/30

(52) U.S. Cl. CPC G16C 20/70; G16C 20/30

(57) ABSTRACT

A method comprising: receiving a plurality of signals representing spectral emissions resulting from an interaction between a laser field and a respective plurality of analytes, wherein at least some of the analytes comprise multi-center chiral molecules; at a training stage, training a machine learning model on a training set comprising: (i) the plurality of signals, and (ii) labels associated with a configuration of a chirality in each of the plurality of analytes; and at an inference stage, applying the machine learning model to a target signal representing spectral emission associated with a target analyte comprising a multi-center chiral molecule, to determine chiral characteristic of the target analyte.

### CROSS REFERENCE TO RELATED APPLICATIONS

This application claims the benefit of priority of U.S. Provisional Patent Application No. 63/091,515, filed Oct. 14, 2020, entitled “MULTI CHIRAL DETECTION”. The contents of the above are all incorporated herein by reference as if fully set forth herein in its their entirety.

### FIELD OF THE INVENTION

The present invention is in the field of methods for detecting and characterizing chirality of an analyte.

### BACKGROUND OF THE INVENTION

Chirality is a fundamental property of asymmetric systems that is abundantly observed in nature. Its analysis and characterization is of tremendous importance in multiple scientific fields, including particle physics, astrophysics, chemistry, and biology. For example, amino acids are generally chiral, as well as DNA and other biologically active molecules, making molecular chiral spectroscopy a necessity for modern drug design.

It turns out that proteins are often highly selective as to the chirality of their binding partner. The binding affinity of a chiral drug can differ substantially for different enantiomers or diastereomers, and thus when designing a drug to interact with the protein molecules one must consider the stereo-selectivity, and possess thorough knowledge of its chiral state. Due to the high selectivity, the FDA has issued in 1992 guidelines and policies concerning the development of chiral compounds. Chiral spectroscopy is therefore paramount, and novel spectroscopic methods are required to enhance signal strength and resolution, as well as to probe systems with ultrafast chiral dynamics.

The foregoing examples of the related art and limitations related therewith are intended to be illustrative and not exclusive. Other limitations of the related art will become apparent to those of skill in the art upon a reading of the specification and a study of the figures.

### SUMMARY OF THE INVENTION

The following embodiments and aspects thereof are described and illustrated in conjunction with systems, tools and methods which are meant to be exemplary and illustrative, not limiting in scope.

There is provided, in an embodiment, a system comprising at least one hardware processor; and a non-transitory computer-readable storage medium having stored thereon program instructions, the program instructions executable by the at least one hardware processor to: receive a plurality of signals representing spectral emissions resulting from an interaction between a laser field and a respective plurality of analytes, wherein at least some of the analytes comprise multi-center chiral molecules, at a training stage, train a machine learning model on a training set comprising: (i) the plurality of signals, and (ii) labels associated with a configuration of a chirality in each of the plurality of analytes, and at an inference stage, apply the machine learning model to a target signal representing spectral emission associated with a target analyte comprising a multi-center chiral molecule, to determine chiral characteristic of the target analyte.

There is also provided, in an embodiment, a method comprising: receiving a plurality of signals representing spectral emissions resulting from an interaction between a laser field and a respective plurality of analytes, wherein at least some of the analytes comprise multi-center chiral molecules; at a training stage, training a machine learning model on a training set comprising: (i) the plurality of signals, and (ii) labels associated with a configuration of a chirality in each of the plurality of analytes; and at an inference stage, applying the machine learning model to a target signal representing spectral emission associated with a target analyte comprising a multi-center chiral molecule, to determine chiral characteristic of the target analyte.

There is further provided, in an embodiment, a computer program product comprising a non-transitory computer-readable storage medium having program instructions embodied therewith, the program instructions executable by at least one hardware processor to: receive a plurality of signals representing spectral emissions resulting from an interaction between a laser field and a respective plurality of analytes, wherein at least some of the analytes comprise multi-center chiral molecules; at a training stage, train a machine learning model on a training set comprising: (i) the plurality of signals, and (ii) labels associated with a configuration of a chirality in each of the plurality of analytes; and at an inference stage, apply the machine learning model to a target signal representing spectral emission associated with a target analyte comprising a multi-center chiral molecule, to determine chiral characteristic of the target analyte.

In some embodiments, the plurality of signals are labeled with the labels.

In some embodiments, the laser field is locally chiral at the interaction.

In some embodiments, the laser field maintains the local chirality within all of an interaction region with each of the plurality of analytes and the target analyte.

In some embodiments, the laser field exhibits any one of the following symmetry properties: static reflection symmetry; dynamical reflection symmetry; dynamical inversion symmetry; dynamical improper rotational symmetry; and lack of inversion, reflection, and improper-rotation symmetry.

In some embodiments, the laser field is generated by illuminating at least two laser beams non-collinearly, wherein at least one of the following is controlled: (i) one or more of the wavelengths of the laser beams, and (ii) one or more of the polarizations of the laser beams.

In some embodiments, the signals represent an intensity of the spectral emissions.

In some embodiments, the signals represent one of ellipticity and polarization handedness of the spectral emissions, or a combination thereof.

In some embodiments, the laser field has different handedness in different sections of the interaction region.

In some embodiments, the spectral emission is a harmonic spectral emission resulting from a high harmonic generation process between the laser field and each of the plurality of analytes and the target analyte.

In some embodiments, the spectral emission is a harmonic spectral emission resulting from a low-order harmonic generation process between the laser field and each of the plurality of analytes and the target analyte.

In some embodiments, each of the plurality of analytes and the target analyte is within a liquid, a solution, a solid or a gas sample.

There is further provided, in an embodiment, a system comprising at least one hardware processor; and a non-transitory computer-readable storage medium having stored thereon program instructions, the program instructions executable by the at least one hardware processor to: obtain reference data comprising a plurality of reference signals representing spectral emissions resulting from an interaction between a laser field and a reference analyte comprising a chiral molecule, wherein molar concentrations of stereo-isomers in the analyte are known, obtain target data comprising a plurality of target signals representing spectral emissions resulting from an interaction between a laser field and a target analyte comprising the specified chiral molecule, calculate reference phase data with respect to each of the reference signals, derive target phase data with respect to the target signals, by applying an optimization algorithm which minimizes an error between the target signals and the reference signals, based, at least in part, on the calculated reference phase data, and reconstruct molar concentrations of stereo-isomers in the target analyte, based, at least in part, on the target phase data.

There is further provided, in an embodiment, a method comprising: obtaining reference data comprising a plurality of reference signals representing spectral emissions resulting from an interaction between a laser field and a reference analyte comprising a chiral molecule, wherein molar concentrations of stereo-isomers in the analyte are known; obtaining target data comprising a plurality of target signals representing spectral emissions resulting from an interaction between a laser field and a target analyte comprising the specified chiral molecule; calculating reference phase data with respect to each of the reference signals; deriving target phase data with respect to the target signals, by applying an optimization algorithm which minimizes an error between the target signals and the reference signals, based, at least in part, on the calculated reference phase data; and reconstructing molar concentrations of stereo-isomers in the target analyte, based, at least in part, on the target phase data.

There is further provided, in an embodiment, a computer program product comprising a non-transitory computer-readable storage medium having program instructions embodied therewith, the program instructions executable by at least one hardware processor to: obtain reference data comprising a plurality of reference signals representing spectral emissions resulting from an interaction between a laser field and a reference analyte comprising a chiral molecule, wherein molar concentrations of stereo-isomers in the analyte are known; obtain target data comprising a plurality of target signals representing spectral emissions resulting from an interaction between a laser field and a target analyte comprising the specified chiral molecule; calculate reference phase data with respect to each of the reference signals; derive target phase data with respect to the target signals, by applying an optimization algorithm which minimizes an error between the target signals and the reference signals, based, at least in part, on the calculated reference phase data; and reconstruct molar concentrations of stereo-isomers in the target analyte, based, at least in part, on the target phase data.

In some embodiments, the specified chiral molecules has n chiral centers, and wherein the reference data comprises at least 2ⁿ⁺¹−1 the signals.

In some embodiments, the reference data comprises: (i) signals associated with each stereo-isomer of the chiral molecule; and (ii) signals associated with mixture of each of the stereo-isomers and a reference one of the stereo-isomers.

In some embodiments, the reference phase data comprises a sign of a relative phase data with respect to the mixtures.

In some embodiments, the program instructions are further executable to measure, and the method further comprises measuring, a plurality of harmonics with respect to each of the signals.

In some embodiments, the program instructions are further executable to measure, and the method further comprises measuring, with respect to a chiral molecule having n chiral centers, at least 2ⁿ−1 harmonics.

In some embodiments, only a subset of the stereo-isomers is analyzed.

In addition to the exemplary aspects and embodiments described above, further aspects and embodiments will become apparent by reference to the figures and by study of the following detailed description.

### BRIEF DESCRIPTION OF THE DRAWINGS

Exemplary embodiments are illustrated in referenced figures. Dimensions of components and features shown in the figures are generally chosen for convenience and clarity of presentation and are not necessarily shown to scale. The figures are listed below.

FIG. 1 shows an exemplary chiral system of four chiral molecules that have the same atomic constituents, but a different ‘geometrical’ organization of the functional groups around the carbon centers;

FIG. 2 is a schematic illustration of an exemplary system for obtaining a spectral line of nonlinear harmonic emission from a molecule, according to certain embodiments of the present disclosure;

FIG. 3 is a flowchart of the functional steps in a process for detecting and determining the configuration of multi-center chiral molecules, according to some embodiments of the present disclosure;

FIG. 4 is a flowchart of the functional steps in an alternative process for detecting and determining the configuration of multi-center chiral molecules, according to some embodiments of the present disclosure;

FIGS. 5A-5B show preliminary numerical results from multiple calculations which simulate nonlinear response of the exemplary chiral system similar to those shown in FIG. 1, as it interacts with the optical setup of the system presented in FIG. 2, according to certain embodiments of the present disclosure;

FIG. 6 shows preliminary experimental results for a chiral molecule with one chiral center (Limonene), according to certain embodiments of the present disclosure; and

FIGS. 7A-7B show preliminary numerical results of chiral mixture reconstruction using the present reconstruction algorithms, according to certain embodiments of the present disclosure.

### DETAILED DESCRIPTION OF THE INVENTION

The present invention, in some embodiments thereof, provides a method and a system for identifying chirality of an analyte. In some embodiments, the present disclosure provides for detecting and determining the configuration of multi-center chiral molecules.

Many molecules in the pharmaceutical industry are in fact chiral, i.e., the present a lack of inversion symmetry of the molecule (similar to “left” and “right” human hands, which lack the inversion symmetry). Molecules with differing chirality interact differently with the amino acids in the human body, and thus different chirality of a specific drug will dramatically influence the operation as well as the efficiency of the drug. For a molecule with a single chiral center, the center is characterized with either (R) or (S) chirality (similar to right- and left-handedness), and the pharmaceutical industry has the machinery to characterize the concentrations of each enantiomer ((R) and (S) chirality).

Known detection methods typically involve optical rotation of light, however, these methods have poor signal to noise ratio, and are thus capable of characterizing only one- or two-center molecules. The main reason for the relatively poor SNR is that current technology relies mostly on the magnetic interaction of light and matter which is relatively weak, such that the signal is deeply immersed in background, with SNR limited to less than 0.001 in certain applications. Such low SNR makes it very difficult to reach high accuracy in single-center chiral molecules, and even harder to tackle the problem of two-center chiral molecules.

With the progress of pharmaceutical medicine, the complexity of the molecule increases rapidly over time, and there is a significant need for characterizing multi-center chiral molecules (e.g., with 3 centers and more).

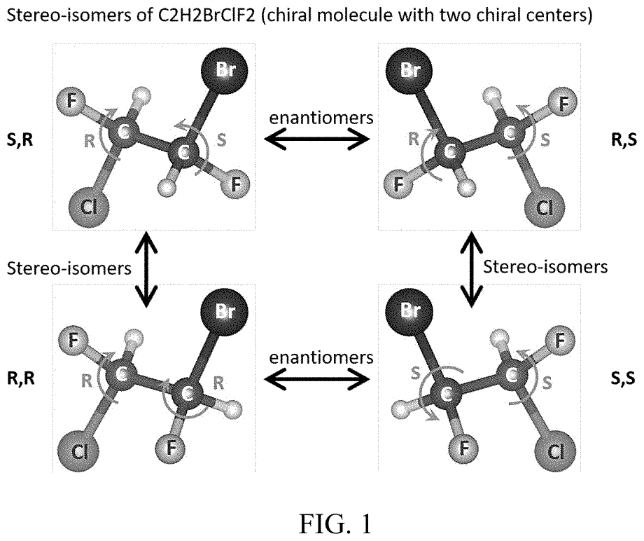

Accordingly, in some embodiments, the present disclosure provides for the identification and classification of chiral compounds (e.g., in liquid, gas, or solid phase), and particularly, compounds that are also comprised of molecules with more than one active chiral center. FIG. 1 shows such an exemplary chiral system of four chiral molecules that have exactly the same atomic constituents (C2H2BrClF2), but a different ‘geometrical’ organization of the functional groups around the carbon centers. The molecule in FIG. 1 comprises 4 stereo-isomers, because it has N=2 chiral centers. The illustration shows the relations of the different stereo-isomers, i.e., which are enantiomers of one another (mirror images), and which are dia-stereo-isomers. As can be seen, each carbon center is connected to four distinct chemical groups, and hence it is a center of chirality (or ‘stereocenter’), making the molecules chiral. These four molecules are termed stereo-isomers of one another, because they have identical constituents and structure, and differ only by a re-organization of the orientations of the functional groups around the chiral centers. Different stereo-isomers are denoted according to the handedness (e.g., right-handedness or left-handedness) around the chiral centers, denoted by the letters (R) and (S) in FIG. 1.

As expected, the separation and identification of such molecules is extremely difficult, because they have exactly the same specific weight, as well as very similar other physical and chemical properties. In fact, the only difference between these molecules arises upon their interaction with other chiral molecules (as in the chemical reactions that occur in the human body), or when interfered with light.

Accordingly, in some embodiments, the present disclosure provides for a process which determines the chirality of compounds with multiple chiral centers, based on shining an intense laser field onto a molecular (or solid) medium, wherein the medium reacts to this laser field by emitting new photons.

As used herein the terms ‘analyte’ or ‘mixture’ refer to a material of interest that may be present in a sample. In some embodiments, the analyte or mixture comprises a chiral molecule or molecular gas, liquid solution or solid. In some embodiments, the analyte or mixture comprises an achiral molecule or molecular gas, liquid solution or solid. In some embodiments, the analyte or mixture comprises a racemic mixture. Suitable analytes and mixtures according to the present invention include organic molecules, catalysts, biocatalysts, bio-molecules such as polypeptides, proteins, enzymes, ribozymes, or the like, or mixtures or combination thereof. In some cases, the term medium may be used herein to depict the analyte and the material thereof.

As used herein, a ‘chiral’ molecule is a molecule that is not superposable on its mirror image (i.e., the molecule does not possess a plane of symmetry). Most chiral organic molecules contain one or more stereogenic centers which are carbon atoms that are bonded to 4 different groups. The pair of non-superimposable mirror images are generally referred to as enantiomers. A solution, mixture, or substance that comprises an excess of an enantiomer is often referred to as being optically active. That is, the plane of polarization of a beam of plane polarized light passed through the solution or mixture containing an excess of one chiral form of a molecule is typically rotated. Specifically, an enantiomer that rotates the plane of polarized light clockwise (to the right) as seen by an observer is dextrorotatory (indicated as D or +) and an enantiomer that rotates the plane of polarized light counterclockwise (to the left) is levorotatory (indicated as L or −). Because of this optical activity, enantiomers are often referred to as optical isomers or optically active. A mixture of equal number of both enantiomers is called a “racemic” mixture or a “racemate.”

In some embodiments, the chiral characteristic of an analyte can be determined in accordance with the symmetry breakings.

As used herein, a molecule's configuration is the spatial arrangement of the atoms of a chiral molecular entity (or group) and its stereochemical description e.g. (R) or (S), referring to Rectus, or Sinister, respectively. As used herein, (R) and (S) denote enantiomers, wherein each chiral center may be labeled as (R) or (S) according to a system by which its substituents are each assigned a priority, according to the Cahn-Ingold-Prelog priority rules (CIP), based on atomic number.

As used herein, a spectral line may be a dark or bright line in an otherwise uniform and continuous spectrum, resulting from emission or absorption of light. A spectral line typically extends over a range of frequencies. In some cases, the spectral line can be a narrow line with narrow range of frequencies. In some cases, the spectral line can be a broad line with a broad range of frequencies. In some embodiments, the obtained spectral line is a result of the emission of non-linear harmonics from a chiral analyte in a sample. In some embodiments, when the analyte in a sample is achiral or racemic, no non-linear harmonics are emitted. In some embodiments, the spectral line obtained is correlated to the magnitude of the enantiomeric excess in a sample.

In some embodiments, the present method provides a (R)/(S) chiral sensitivity. In some embodiments, the present method provides chiral/achiral sensitivity. In some embodiments, there is provided a method to determine the chirality of an analyte in a sample. In some embodiments, there is provided a method to differentiate between the (R) and (S) chirality of an analyte in a sample. In some embodiments, there is provided a method to determine if an analyte in a sample is chiral or achiral.

In some embodiments, the method relies mainly on electric-dipole interactions. In some embodiments, the method is not dependent on the interaction with the magnetic field of the illuminating laser.

In some embodiments, the present disclosure uses the generation of high harmonics through ultra-short laser pulses, which are shaped such that the chiral interaction is through the electric dipole (in contrast with the magnetic dipole in known methods), and is thus considerably stronger.

Accordingly, in some embodiments, the present disclosure builds on the concept of using high harmonics and chiral light in order to detect chiral centers of molecules, as fully disclosed by the present inventors in International Patent Application No. PCT/IL2019/050709, filed Jun. 25, 2019, the contents of which are incorporated herein in their entirety.

In some embodiments, the present disclosure provides for one or more novel reconstruction algorithms for the analysis of the detected signal, to solve the inverse problem of configuration determination. In some embodiments, the present algorithms incorporate deep learning techniques.

In order to analyze the specific configuration of a test solution, one needs to solve an inverse problem. From calculations that the present inventors have performed, it turns out that different stereo-isomers in the solution emit fields with different phases to a specific harmony. These fields in a specific harmony are then summed up and squared to give the resulting intensity at the specific harmony. The result, for a specific configuration, yields:

$$I ∝ {‖ {\sum }_{i} {n}_{i} {g}_{i} {e}^{i {α}_{i}} ‖}^{2}$$

with gᵢ, αᵢ being the amplitude and phase of a specific stereo-isomer i to the incident laser field. In a two-center molecule, for example, i can take the values 0, 1, 2, 3 which correspond to RR, SS, RS, SR configurations. The configuration of the solution is then given by the vector nᵢ of concentrations. In order to determine gᵢ, αᵢ, multiple experiments may be conducted with varying known concentration vectors, nᵢ, allowing two different algorithms to solve the inverse problem.

The understanding of this process is novel, and allows, in some embodiments, for the present disclosure to provide for novel algorithms to analyze the molecule configuration. In some embodiments, a first algorithm of the present disclosure receives as input measurements of known concentrations, and then uses a steepest descent method to estimate the concentration of each of the different stereo-isomers in the solution. In some embodiments, a second algorithm of the present disclosure comprises a machine learning model trained on a dataset comprising measurements in different known concentrations prior to the testing, wherein the measurements are labeled with the known concentrations. In some embodiments, at an inference stage, the trained machine learning model may be applied to a target measurement of a target solution, to determine the stereo-isomers concentration in the target solution.

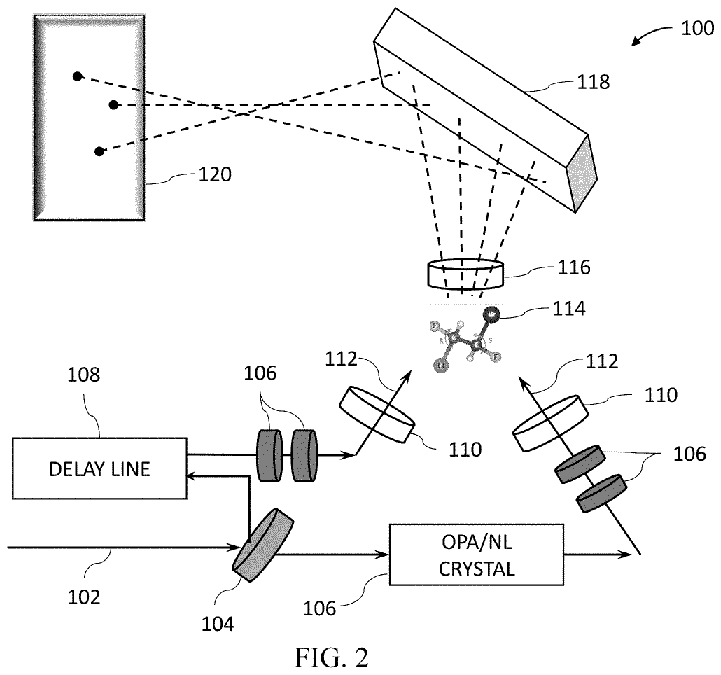

FIG. 2 is a schematic illustration of an exemplary system 100 for obtaining a nonlinear harmonic spectral emission from a molecule. In some embodiments, system 100 comprises a two or more laser beam geometry, where the beams 112 are non-colinear. The beams also have a different main frequency component that can be generated using several methods (e.g., an OPA or a nonlinear crystal 106). Both beams 112 are focused, such that they overlap in space and time, into the chiral medium 114, which may be a cuvette or any other capsule that holds the mixture, or a mechanism that allows the liquid to flow freely while analyzed. Due to the interaction with the beams, the medium emits new light frequencies that are measured in a spatially and frequency-resolved way, e.g., by a spectrometer or camera 120. System 100 may further comprise one or more delay lines 108, lenses 110, waveplates 106, polarizers 116, and gratings 118. The various components of system 100 as shown in FIG. 2 may be arranges in multiple ways in relative to one another, and may utilize varying relative angles, polarizations, frequencies, intensities, and the like, to obtain the best signals.

Accordingly, in some embodiments, the present disclosure provides for using two or more beams of intense laser light that are simultaneously directed at the sample. The opening angles, frequency ratios, and polarization states of the beams are selected so as to generate an electromagnetic light field that exhibits a unique symmetry in structure in its time-dependent polarization. In some embodiments, by having the two beams in a non-colinear geometry (i.e., at an angle to one another), and operating with different frequencies, the resulting non-linear response of the medium is able to discriminate between all types of stereo-isomers, regardless of how many chiral centers they are comprised of.

In some embodiments, the propagation direction of the first and second laser beams 112 form an angle, referred to as non-collinear configuration. In some embodiments, the angle of incidence of the first and second laser beams 112 is in the range of 0° to 90°, including any range therebetween.

In some embodiments, additional beams may be added to the system, e.g., a third and, in some embodiments, a fourth beam, in order to further break the symmetry of the light pulse. Such beams may possess different central frequencies, as well as different polarizations and spatial field distribution. Such fields may increase or decrease the chiral sensitivity of the laser light to the chirality of the solution.

In some embodiments, the propagations of the laser beams 112 overlap in space. In some embodiments, the propagations of the laser beams 112 overlap in time. In some embodiments, projecting the first laser beam and the second laser beam occurs at the same time or different time intervals. The frequencies (ωᵢ=2πc/λᵢ were λ is the wavelength and c is the speed of light) are determined by several consideration: the ratio between the two frequencies ω₁/ω₂=λ₂/λ₁ needs to be odd:odd for achieving dynamic reflection or dynamical inversion symmetries. The frequencies also should be far from resonance of the analyte (for most cases 800-2500 nm is far from any resonance). Another practical consideration is to have a strong enough source for the beams (which is available in the range of 400-2200 nm). For example, 1333 and 800 nm for 3/5 ratio or 1200 and 800 nm for 2/3 ratio can be used.

In some embodiments, the first laser beam has a wavelength of 800 nm. In some embodiments, the second laser beam has a wavelength of 400, 1200 or 1333 nm.

In some embodiments, the first laser beam and the second laser beam have a frequency ratio in the range of x:y to x:y 1:1, 1:2, 2:3, 3:5

In some embodiments, the first laser beam and the second laser beam have an odd:odd frequency ratio. Non-limiting examples of odd:odd frequency ratios include 1:3, 1:5, 1:7, 3:1, 3:3, 3:5, 3:7. In some embodiments, the first laser beam and the second laser beam have an even:odd frequency ratio. Non-limiting examples of even:odd frequency ratio include 2:1, 2:3, 2:5, 4:1, 4:3, 4:5, 4:7.

In some embodiments, the first laser beam and the second laser beam are co-planar.

In some embodiments, the first laser beam and the second laser beam have the same frequency. In some embodiments, the first laser beam and the second laser beam have the same frequency and are co-planar. In some embodiments the first laser beam and the second laser beam have different frequencies.

In some embodiments, a polarization state of the first laser beam is linearly, elliptically, or circularly polarized. In some embodiments, a polarization state of the second laser beam is linearly, elliptically, or circularly polarized. In some embodiments, the first laser beam and the second laser beam have the same polarization state. In some embodiments the first laser beam and the second laser beam have a different polarization state.

In some embodiments, the ratio between the wavelength of the first laser beam and the wavelength of the second laser beam is practically the same. In some embodiments, the first laser beam and the second laser beam originate from the same source. In some embodiments, the first laser beam and the second laser beam originate from a different source. In some embodiments, the source is a laser beam. In some embodiments, the laser beam is split into the first laser beam and the second laser beam.

In some embodiments, the second laser beam is originated through an optical parametric amplifier (OPA) 106. In some embodiments, the OPA 106 converts the frequency of the second laser beam into chosen values, obtaining odd or even frequency ratio with respect to the first laser beam.

In some embodiments, the signal is found in the intensity of the emitted spectrum if the optical set-up is chosen to be ‘locally chiral’ (i.e., lacking any particular reflection-based symmetries), or in the polarization states of the emission if the optical set-up is purposefully chosen to exhibit some reflection-based symmetry. This emission (which is denoted as the molecular ‘non-linear response’) can be measured directly by using various imaging modalities, e.g., cameras and/or spectrometers. The non-linear response permits separating the different constituents (and molar ratios) of a generic and unknown chiral compound, however, typically it does not differentiate between stereo-isomers of a chiral molecule.

In some embodiments, the non-linear emission in this special two-beam configuration may be determined from the pure samples of chiral stereo-isomers, to generate reference data. Each isomer has a unique spectral signature (molecular fingerprint) that is sensitive to the orientation of the functional groups around its chiral centers. The signal difference in the present method can reach 100%, and is normally on the order of tens of percent. The extremely large signal means that it is possible to determine the constituents of a compound to a very high accuracy, as well as sense and analyze chirality of novel molecules that are in interest of the medical industry, which are standardly very difficult to test.

Upon taking a generic measurement from an unknown mixture of stereo-isomers, the molar concentrations of each isomer can be determined by comparing to the reference data, and by using a reconstruction algorithm. This measurement is single-shot, and extremely fast. It is also, in principle, general to any type of molecule, regardless of its size, constituents, solubility, toxicity, phase of matter, etc. These advantages make our technology extremely appealing for industry use, as it both solves an outstanding problem, as well as presenting a solution which is robust and effective.

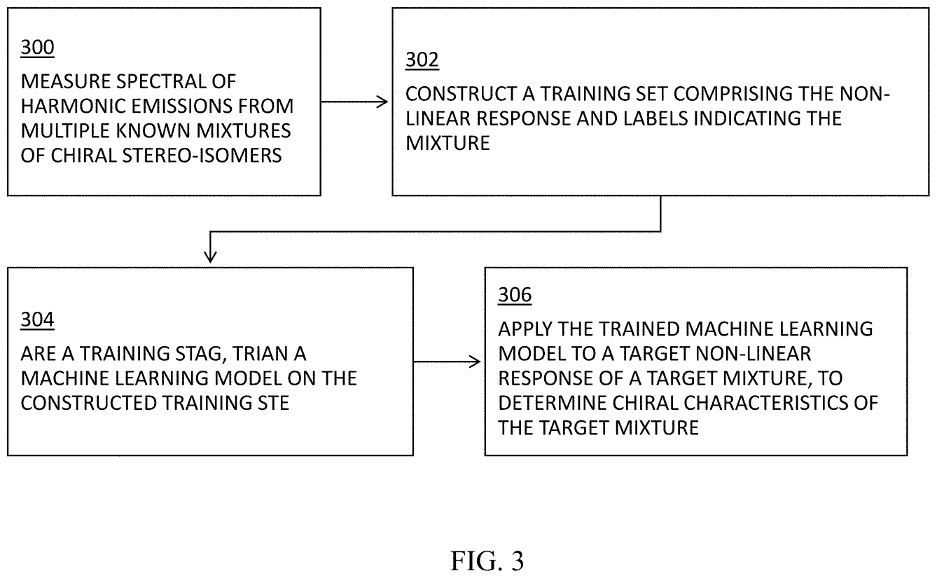

FIG. 3 is a flowchart of the functional steps in a process for detecting and determining the configuration of multi-center chiral molecules, according to some embodiments of the present disclosure.

In some embodiments, at step 300, spectral signals are measured with respect to a plurality of analytes or mixtures having known concentrations of stereo-isomers. In some embodiments, the spectral signals comprise, e.g., nonlinear harmonic spectral emission from a molecule interrogated by an optical system, such as system 100 in FIG. 2. In some embodiments, at least some of the plurality of mixtures comprise multi-center chiral molecules.

In some embodiments, these measurements will be used in constructing a training dataset at next step 302. Accordingly, in some embodiments, in order to increase the size of the training set and make it more robust, a plurality of measurements may be measured with respect to each mixture, by, e.g., through different input polarizations, different input wavelengths, as well as through probing different output harmonic frequencies of the spectral signals.

Accordingly, in some embodiments, with respect to each mixture, the present disclosure may provide for measuring multiple output frequencies (e.g., harmonies), generated through different input polarization scenarios, and using different wavelengths.

In some embodiments, these different measurements may be taken with respect to each specific stereo-isomer i of the mixture or analyte. For example, in a two-center molecule, these stereo-isomers correspond to RR, SS, RS, SR configurations. In a mixture with n centers, there will be 2ⁿ combinations. The configuration of the solution is then given by the vector nᵢ of concentrations, wherein multiple experiments may be conducted with varying known concentration vectors, nᵢ.

In some embodiments, at step 302, the present disclosure provides for constructing a training set comprising the measured signals from each of the mixtures, labelled by labels indicating the respective known concentration of each mixture.

In some embodiments, at step 304, a machine learning model may be trained using the training set constructed at step 302.

Finally, at step 306, the trained machine learning model may be applied to a target mixture, to determine the chiral characteristics of the target mixture. In some embodiments, the target mixture comprises a multi-center chiral molecule.

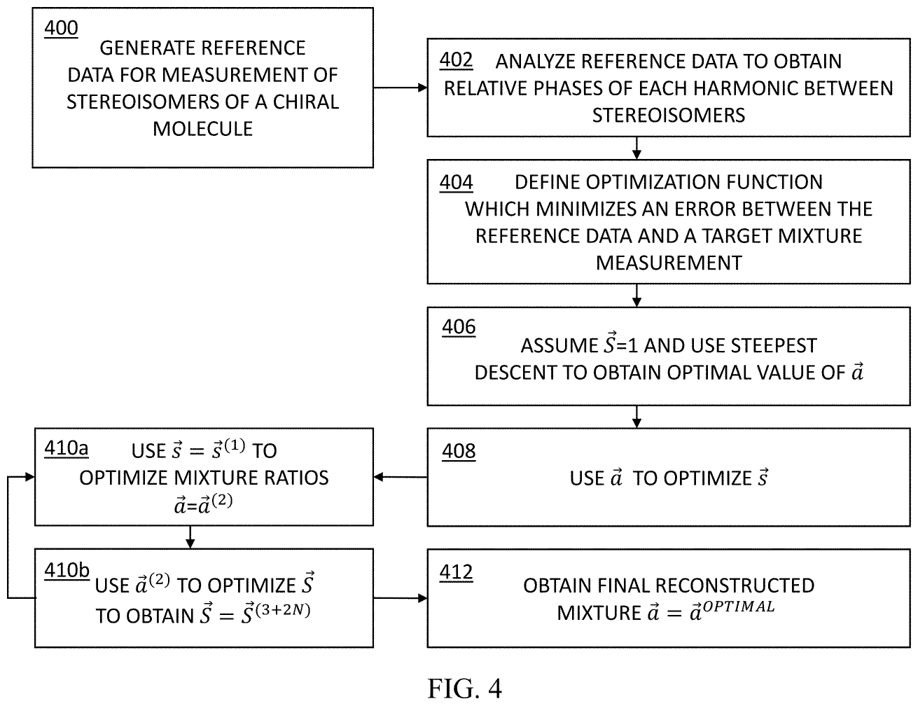

FIG. 4 is a flowchart of the functional steps in an alternative process for detecting and determining the configuration of multi-center chiral molecules, according to some embodiments of the present disclosure.

In some embodiments, the present disclosure provides for an algorithm which reconstructs molar concentrations in a mixture of chiral molecules, from which a single measurement is taken using the technique disclosed herein above, e.g., with reference to FIG. 2. Similarly to the training set in the forementioned analysis, the approach relies on the existence of reference data from the pure stereoisomers, however utilizes a steepest descent optimization approach for reaching an optimal solution, even in the presence of noise.

In some embodiments, the present algorithm may be particularly useful when both reference data and measured data are noisy.

The purpose of the present algorithm is to fully characterize the stereoisomers configuration of an unknown mixture. For this, the intensity of a few harmonics emitted from the mixture is measured with respect to a specific beam arrangement. Each of the harmonics will be emitted with a specific phase from a specific stereoisomer. This means that the intensity of a specific harmonic will be:

I^{(h)}=∥ΣᵢEᵢe^{jα}^{ᵢ}∥,

where Eᵢ is related to the strength of a specific stereoisomer, and αᵢ is the relative phase of the emitted light from the i^{th} harmonic. In order to find the relative strength of each stereoisomer, which will lead to the full configuration, the relative phases are analyzed and retrieved in advance. Thus, a set of calibration measurements may be provided, which will produce the reference data, and then the measurement of the unknown mixture to be analyzed.

### Generating Reference Data

With continued reference to FIG. 4, in some embodiments, at step 400, the present algorithm provides for generating reference data needed to perform the reconstruction. It is assumed that a chiral molecule with N chiral centers in considered. This means that in total there are 2^{N} different stereoisomers whose molar concentration in the solution needs to be reconstructed. For this, reference data from 2^{N+1}−1 measurements is required. 2^{N} measurements from each of the pure samples of the stereoisomers recording the power of the harmonics (possibly also along specific polarization axes), and additional 2^{N}−1 measurements from 50/50 mixtures of each of the stereoisomers with a single stereoisomer that is chosen to be used as reference. The 50/50 mixture measurements are later used to reconstruct the values of the harmonics relative phase, αᵢ. This particular stereoisomer may be labeled with an index reference “1.” In each measurement, several harmonic lines are measured, and when more harmonics are measured the reconstruction error reduces. At the minimum, at least 2^{N}−1 harmonics have to be measured to have enough data for a full reconstruction. A measurement is performed at a single beam geometry of chiral light, and should be exactly the same geometry that is used in each of the measurements that follow, both for reference data, and from the unknown mixture for reconstruction. In some embodiments, additional redundant data from more beam geometries may be measured and used to reduce reconstruction errors.

In some embodiments, data from different harmonics may be complemented by data from different input polarizations, in order to complete the full 2^{N}−1 reference set.

In some embodiments, only partial configuration may be required. In that case, not all 2^{N} should be mapped, and the reference data will be prepared accordingly. i.e., less reference measurements are required in order to complete a partial analysis of the configuration.

In a specified example, in the case of N=2, a chiral molecule with 2 chiral centers has 2^{N}=4 stereoisomers, labeled “1,” “2,” “3,” and “4.” One must measure at least 2^{N}−1=3 harmonic lines (e.g., harmonic lines h=2,3,4), for a total of 2^{N+1}−1=7 measurements. Out of these 7 measurements, 4 measure the harmonic lines emitted from the pure stereoisomer samples, and an additional 3 measure harmonic lines emitted from the 50/50 mixtures of stereoisomers “1” and “2,” “1” and “3,” and “1” and “4.” The data can be labeled for convenience as follows: Iⱼ^{(h)} indicates the measured power of the hth harmonic from the pure sample of the jth stereoisomer. I₁ⱼ^{(h)} indicates the measured power of the hth harmonic from the 50/50 mixture of the jth stereoisomer combined with the 1st stereoisomer.

### Obtaining Relative Phases of Harmonics From Reference Data

In some embodiments, at step 402, the reference data may then be analyzed to obtain the relative phases of each harmonic from each pure stereoisomer sample with respect to the sample “1,” which is labeled ϕ₁ᵢ^{(h)}. From the given set of measurements, this can only be done up to a sign (i.e., only the absolute value |ϕ₁ᵢ^{(h)}| can be recovered). Technically, the sign can be recovered by additional reference measurements of mixtures between all of the stereoisomers, i.e. by performing 2^{N-1}! total measurements. Alternatively, the sign of the phase may be reconstructed using the algorithm itself instead. Using the additional reference data would simplify the algorithm and reduce reconstruction errors, but also increases substantially the amount of measurements required.

In some embodiments, the actual phases are reconstructed as follows:

$${ϕ}_{1 i}^{( h )} = arcos ( \frac{4 {I}_{1 i}^{( h )} - {I}_{1}^{( h )} - {I}_{j}^{( h )}}{2 \sqrt{{I}_{1}^{( h )} {I}_{j}^{( h )}}} )$$

### The Reconstruction Algorithm

Upon obtaining the reference data and relative phases of harmonics, in some embodiments, the present algorithm may be used to reconstruct the molar concentrations of different stereoisomer constituents in a chiral mixture, from which a measurement is performed (measurement refers to the procedure described above for the reference data). The molar concentrations are labeled as aᵢ, where “i” is the index of the stereoisomer running from “1” to 2^{N}, wherein M=2^{N}.

The molar concentrations formally uphold the following constraints that are applied in the algorithm: 1=Σᵢ₌₁^{M}aᵢ, which is used to determine: a_{M}=1−Σᵢ₌₁^{M-1}aᵢ (which reduces the number of parameters to reconstruct from M to M−1). Also, each aᵢ upholds: 0≤aᵢ≤1.

The desired operation of the algorithm is then to reconstruct the values of aᵢ from measured data from an unknown target mixture, which is labeled as Iₘᵢₓ^{(h)}, and the previously-configured relative phases.

In some embodiments, at step 404, in order to achieve this objective, a function may be defined which minimizes the absolute error between the measured Iₘᵢₓ^{(h)}, and the one that can be constructed from the reference data given aᵢ:

$${f}_{tar}^{( h )} ( \vec{a} , {\vec{s}}^{( h )} ) = {I}_{mix}^{( h )} - \vert {\sum }_{j = 1}^{M} {a}_{j} \sqrt{{I}_{j}^{( h )}} {e}^{i {ϕ}_{1 j}^{( h )} {s}_{1 j}^{( h )}} \vert$$

where s₁ⱼ^{(h)} is the sign of the relative phase between stereoisomer “1” and “j” in the reference data for harmonic h, which can take values ±1 and is unknown, and is compactly labeled as \vec{s}^{(h)}. The value ϕ₁₁^{(h)}=0. In addition, aᵢ are unknowns and are the property of interest, denoted compactly by \vec{a}.

From this target function for each harmonic order, the full target function that averages the error over all harmonic indices may be constructed:

$${F}_{tar} ( \vec{a} , \vec{s} ) = {\sum }_{h} \vert {f}_{tar}^{( h )} ( \vec{a} , {\vec{s}}^{( h )} ) \vert$$

where \vec{s} now denotes the signs of relative phases for all harmonics and all stereoisomers.

The logic here is now to vary \vec{a} and \vec{s} in order to minimize Fₜₐᵣ, at which point the reconstruction in complete. In some embodiments, this may be achieved using a steepest-descent algorithm as implemented by the MATLAB function “globalsearch,” which searches a global minimum for a given target function. This problem is treated here for simplicity by a separation of variables; first the optimal values of \vec{s}, then \vec{a} are reconstructed until convergence is reached and they no longer vary, or until a minimal iteration criteria is satisfied.

In some embodiments, at step 406, it is assumed that \vec{s}=1 for all harmonic orders and from all stereoisomers, optimizing Fₜₐᵣ⁽⁰⁾(\vec{a})=Fₜₐᵣ(\vec{a},\vec{s}=1) to obtain ideal values of \vec{a}. This may be done by a combined Monte-Carlo type approach, e.g., guessing an initial Nᵢₜₑᵣ=100 random combinations of \vec{a}=\vec{a}_{guess}, and running “globalsearch” from each of these guess values to find the optimal \vec{a}.

In some embodiments, the constrains for aᵢ discussed above may be employed, and an additional ensemble of guess points may be created around each \vec{a}_{guess}, only the best of which are propagated to be fully optimized by “globalsearch”. Out of all of these optimizations, the best value is chosen for the mixture taken as \vec{a}⁽⁰⁾.

In some embodiments, at a next step 408, the values for mixture ratios \vec{a}=\vec{a}⁽⁰⁾ may be used to optimize the target function in terms of the signs of the relative phases, i.e., the function Fₜₐᵣ⁽¹⁾(\vec{s})=Fₜₐᵣ(\vec{a}=\vec{a}⁽⁰⁾,\vec{s}) may be optimized. Because \vec{s} is a vector with values ±1 of length hₘₐₓ×(M−1), it is possible to simply calculate directly Fₜₐᵣ⁽¹⁾ (\vec{s}) for all possible combinations of inputs in \vec{s}. In total, there are exactly hₘₐₓ×(M−2)! calls for the function Fₜₐᵣ⁽¹⁾(\vec{s}), i.e., there are M−1 relative phases, but one of them can be arbitrarily set because the signal intensity is invariant to an operation of complex conjugation, leaving M−2 phases to set with (M−2)! options for ordering but one for each harmonic line. This is computationally much faster than running an optimization algorithm, though it is noted that one may simply apply the same algorithm as above to \vec{s}, which should lead to the same solution. Out of all of these calculations the configuration of \vec{s}=\vec{s}⁽¹⁾ that minimizes Fₜₐᵣ⁽¹⁾(\vec{s}) may be selected.

In some embodiments, at iterative step 410, the step 410a of optimizing d only may be repeated while using \vec{s}=\vec{s}⁽¹⁾, i.e., optimize Fₜₐᵣ⁽²⁾(\vec{a})=Fₜₐᵣ(\vec{a},\vec{s}=\vec{s}⁽¹⁾), using the exact same method as in step 406. This gives an optimal value \vec{a}=\vec{a}⁽²⁾.

This procedure goes on self-consistently in a loop (i.e. all even steps optimize the phase signs, and all odd steps optimize the mixture ratios) until the phase signs do not vary between iterations, leading to the reconstructed value \vec{a}^{optimum}. In practice, it was found that 4 iterations may be required to achieve convergence for tested cases, i.e. \vec{a}^{optimum}=\vec{a}⁽⁴⁾.

In some embodiments, the present disclosure can be configured to identify the chiral characteristics of an analyte, based on symmetry breaking phenomena, wherein a spectral line of a nonlinear harmonic emission resulting from a harmonic generation (e.g., high or low order harmonic generation) on the analyte is measured. In some cases, such a method can produce a signal correlated with a magnitude of the enantiomeric excess in an analyte.

According to some embodiments, there is provided a method for identifying chiral characteristics of an analyte, based on symmetry breaking phenomena, wherein a spectral line of a nonlinear radiation resulting from a wave-mixing nonlinear process causes a polarization density which responds non-linearly to the electric field of the light. In some case where nonlinear radiation results from a wave-mixing nonlinear process, the method and system disclosed herein can be configured to analyze a spectral line with multiple orders.

In some embodiments, both the spectral and spatial information are recorded, either by splitting the information to two detectors, or toggling the information between the two. Spatial imaging of both the near-field and the far-field can be utilized to extract spatial and angular information of the generated harmonics.

In some embodiments, the method and system disclosed herein can employ a detection device, e.g., a spectrometer, designed to receive the spectral line or lines. In some embodiments, the device can be coupled with at least one hardware processor and a non-transitory computer-readable storage medium having program instructions stored thereon, the program instructions executable by the at least one hardware processor to receive, and/or measure, and/or analyze the spectral line of the nonlinear harmonic emission.

In some embodiments, chiral characterization by the method and system of the present disclosure relies solely, or in some cases predominantly, on the spectral line analysis dominantly generated by electric-dipole interaction between the laser and the analyte.

In some embodiments, the present disclosure comprises a step of measuring a characteristic of the spectral line, such as in respect to a predefined measuring model. Measuring the characteristic of the spectral line, allows performing at least part of the analysis processes based on the received spectral line. In some embodiments, measuring a characteristic of an electric field is measuring intensity of the spectral line. In some embodiments, measuring a characteristic of an electric field is measuring any one of ellipticity and polarization handedness of the spectral line, or combination thereof.

In some embodiments, the method and system disclosed herein can be utilized for measuring a characteristic or characteristics of an electric field of the at least one spectral line. In some cases, the characteristic of an electric field of the at least one spectral line can be one or more of the following: (i) wavelengths, and (ii) one or more of the polarizations, (iii) the harmonic number received from the spectral line, (iv) x-polarized high harmonics, (v) x-polarized odd harmonic, (vi) harmonic ellipticity in x-y plane, and (vii) polarized harmonic spectrum.

In some embodiments, the predefined measuring model can comprise, but is not limited to: (i) measuring the level of polarized harmonic spectrum emitted from the chiral/achiral analyte, (ii) measuring harmonic ellipticity according to the harmonic order, wherein the helicity changes sign the analyte's handedness, and (iii) measuring the polarized odd harmonics versus the enantiomeric excess.

In some embodiments, the high harmonic emission on the analyte can be caused by an electric dipole interaction between the laser and the analyte. In some cases, the electric dipole interaction can be generated through focusing two non-collinear laser beams on the analyte. Thus, the analyte can be irradiated with an intense laser field, and the emission spectrum resulting from that laser field can be measured and analyzed, as aforementioned.

In some embodiments of the present invention, the two non-collinear laser beams can be used to induce macroscopic chiral light. Thus, the two non-collinear laser beams generate electric dipole interactions on an analyte which can provide a chiral sensitivity, both in the microscopic response and in the macroscopic scale. The propagation and the phase of the macroscopic chiral light can be photoinduced for the purpose of probing and monitoring the chiral characteristic.

In some embodiments, the focused non-collinear laser pulses induce a three-dimensional vectoral laser field that interacts with the analyte. In some cases, a meta-structure with metasurfaces is illuminated by a laser to induce a three-dimensional vectoral laser field that interacts with the analyte.

In some embodiments, the system and methods disclosed herein can be operated using a layout comprising two non-collinear laser beams set to generate the electric dipole on the analyte required for chiral characteristic processes.

In some cases, the setting of the layout comprising two non-collinear laser beams can harness the fact that chiral analyte inherently breaks certain symmetries, e.g., reflections, inversions, dynamical-reflections, and the like, that are upheld by the pump field. Thus, the setting of the layout can be engineered to illuminate the analyte for generating harmonic emission characterized by diverse symmetries.

In some embodiments, operations required for measuring and analyzing intensities of the spectral line, may be based on the characteristic of the harmonic emission caused by the analyte illumination to define the chiral characteristic of the analyte. In some cases, the characteristic of the harmonic emission caused by the analyte illumination may be considered in, at least part, of the analysis steps.

For example, the vector direction of the electrical field may be considered in the analysis in case the harmonic emission caused by the analyte illumination is characterized by a spherically symmetric ensemble which is invariant under any rotation, reflection, and inversion. Namely, in this exemplary case, the characteristic of the harmonic emission, e.g., the direction of the field, may be considered in the analysis in case the vector direction of the field is dependent on the macroscopic emission of the harmonics.

In some embodiments, the laser pumps can set to exhibit harmonic emission characterized by orientation of enantiomer (R). In some cases, the pumps can set to exhibit harmonic emission characterized by orientation of enantiomer (S). In some embodiments, the laser pumps can set to exhibit harmonic emission characterized by orientation that changes according to the vector direction of the field.

In some cases, a co-propagating single-color can be focused into a metamaterial structure to produce a three-dimensional multi-color pump laser filed. In some other cases, a co-propagating multiple-color beams can be focused into a metamaterial structure to produce a three-dimensional multi-color pump laser field.

The method and system of the present invention can be operated using several settings, based on the architectural and/or configuration variables of the non-collinear laser beam layout. Thus, in some cases, the laser beam architectural and/or configuration variables such as the polarizations of the laser beam, the frequencies thereof, and the angles between the beams, may be changed and/or set, such as to generate the electric dipole interaction with the analyte required for chiral characteristic processes. In some cases, changing and/or setting the architectural and/or configuration variables may be required for the purpose of receiving a number of intensity values of spectral lines which are different from each other.

For example, in one chiral characteristic definition process, a person utilizing a layout comprising two non-collinear laser beams can change the polarization of the at least one of the beams, and/or the angle between the beams, and thereby receive a first spectral line. In this exemplary case, in another chiral characteristic definition process the person can change again the polarization of the at least one of the beams, and/or the angle between the beams and thereby receive a second spectral line.

The term “angle between the beams” refers to the angle measured between two light trajectories of two beams focusing on one point (e.g., the analyte), wherein each light trajectory is defined to be the trajectory of the center of each beam.

In some cases, the system and methods disclosed herein can be employed according to the symmetry breaking in high or low harmonic generation. Thus, architectural and/or configuration variables of the non-collinear laser beam layout can be set for the purpose of receiving diverse symmetry breaking options resulting from the high harmonic generation. For example, the non-collinear laser beam layout can be set to a static reflection symmetry breaking. In some other cases, the non-collinear laser beam layout can be set to a dynamical improper-rotational symmetry breaking.

In some cases, the non-collinear laser beam layout can be set to a dynamic reflection symmetry breaking. In some cases, the non-collinear laser beam layout can be set to a dynamical inversion symmetry breaking.

In some embodiments, a single laser pump can be utilized to generate the harmonic emission. In some cases, a laser beam directed to a metamaterial can be set, to obtain harmonic emission with a spatial field distribution which may be in correlation to the required analysis of the analyte.

In some embodiments, the harmonic emission of photons is obtained by projecting two non-collinear beams comprising a first laser beam and a second laser beam which jointly meet the sample to create the asymmetric light field.

### Numerical & Experimental Results

FIGS. 4A-4B show preliminary numerical results from multiple calculations which simulate nonlinear response of the exemplary chiral system shown in FIG. 1, as it interacts with the optical setup of system 100 presented in FIG. 2. Specifically, FIGS. 4A-4B show the emission spectrum for the different pure compounds in a given configuration of system 100. As shown in FIGS. 4A-4B, different pure stereo-isomers lead to different emission intensities and polarization, with differences ranging in 50-150%. This indicates that the method is indeed suitable to separate different stereo-isomers, regardless of the number of chiral centers in the molecule. The results in FIGS. 4A-4B were achieved using wavelengths of 2400-1200 nm, with a 10 degree opening angle between the two beams 112, 1:1 intensity ratios, and elliptical polarization with ellipticities 0.1. FIG. 4A shows the emitted spectrum (nonlinear response) of the model pure stereo-isomer samples, total response (left), and y-polarized response (right). Clearly different stereo-isomers have their own unique spectral signature to the light field. FIG. 4B shows the calculated resulting chiral-signal between pairs of stereo-isomers in %, normalized from −200 to 200% per standard nomenclature. Very large discrimination signals are obtained (>100%) between each of the stereo-isomers, which forms the basis for a method to separate them.

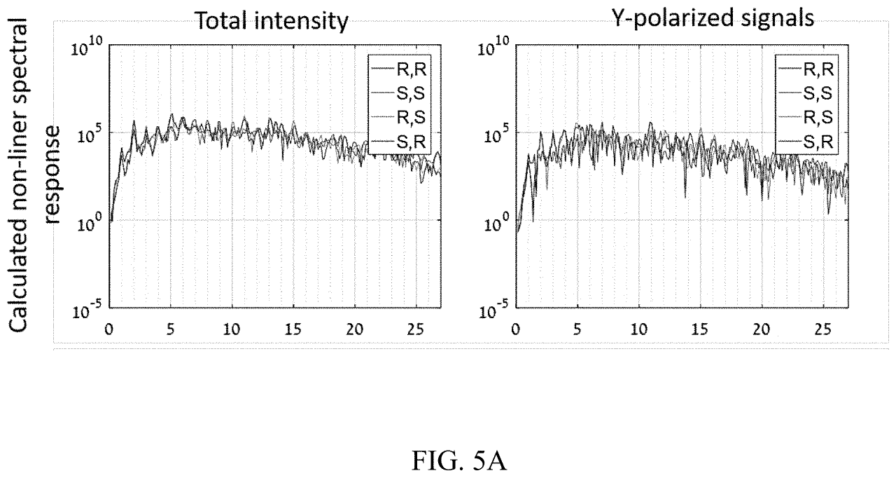

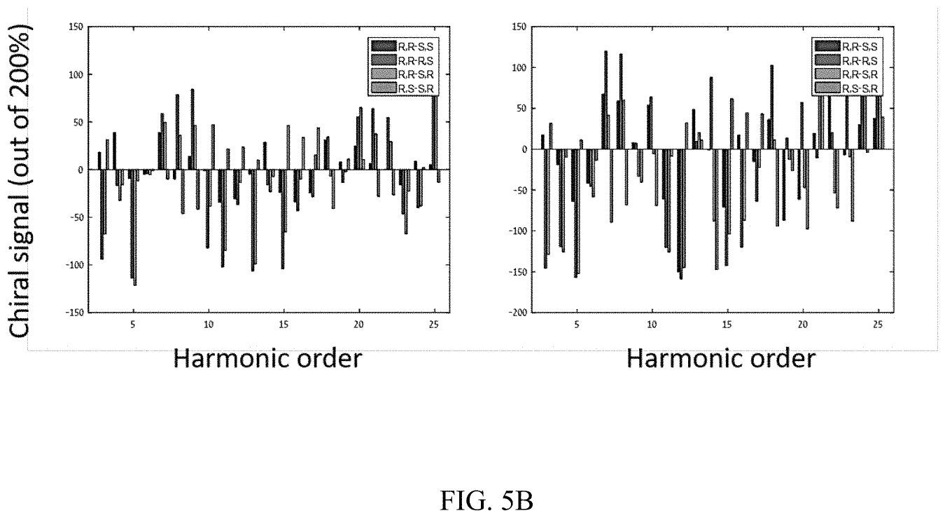

FIG. 5 shows preliminary experimental results for a chiral molecule with one chiral center (Limonene), supporting the results of the theoretical calculations. These experiments are a proof of concept that the method can indeed be used to characterize molecular chirality with a very high accuracy, because the measured chiral signal is a significant at 165%. Specifically, FIG. 5 shows preliminary experimental results from a particular configuration of two optical beams as illustrated in FIG. 2 for chiral Limonene molecules with one chiral center. The figure shows the measured emitted spectrum from both stereo-isomers of the pure samples, showing an unprecedented measured chiral signal of ˜165% between the (R) and (S) enantiomers.

The present inventors then simulated the emission spectrum from an unknown mixture of stereo-isomers. Using the calculated spectrum, and assuming that the reference data from each pure molecule is known, the exact molar ratios of each element in the mixture were reconstructed. This approach is currently implemented with a steepest descent reconstruction algorithm, and also independently with a deep-learning type algorithm. The method can be performed as a single shot measurement that directly outputs the exact structure of the compound from just one measurement.

To test the statistics of this approach, many such mixtures (randomly drawing-up the ratios of the stereo-isomers in the compound) were simulated, and their emission spectrums were measured. This is done 1000 times for each level of noise in the measurements, assuming noise up to 20%. The results show that even with a 5% assumed noise in measurement, smaller than 1% reconstruction errors are obtained on average. This is despite the measurement being single shot, and despite there being four different molecules in the mixtures. Notably, even when the mixture contains equal amounts of enantiomers (e.g., 1:1:2:2 of isomers (R), (R), (S),(S), (R),(S), (S), (R)), accurate reconstructions are obtained. The ability to reconstruct the composition of these particular mixtures is notable because they are on average achiral (because there are equal amounts of the enantiomers), hence such mixtures lead to zero signal with any of the currently used linear-response characterization techniques like optical-rotation or optical absorption spectroscopy.

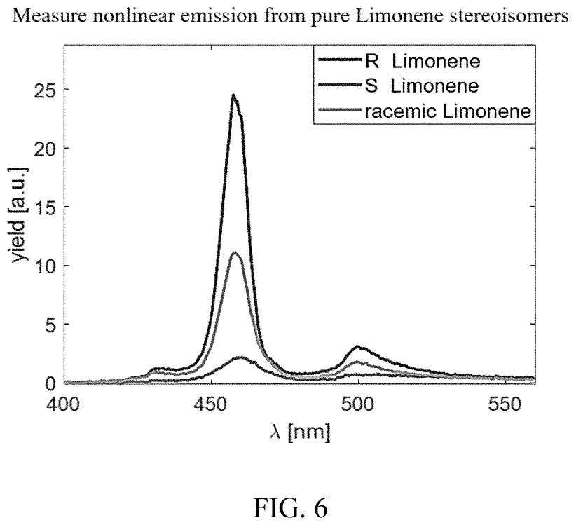

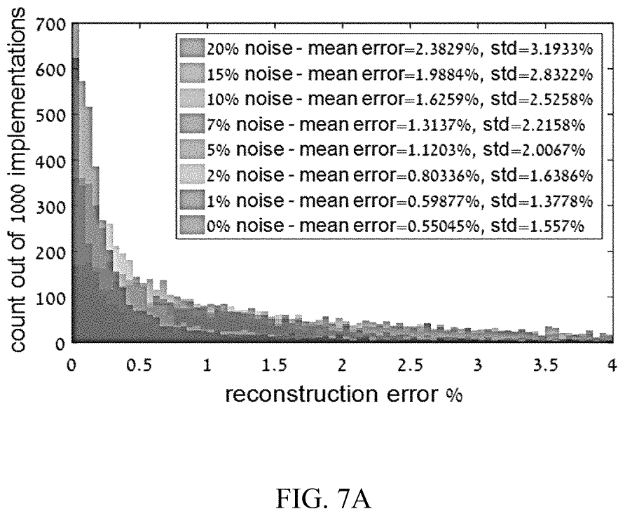

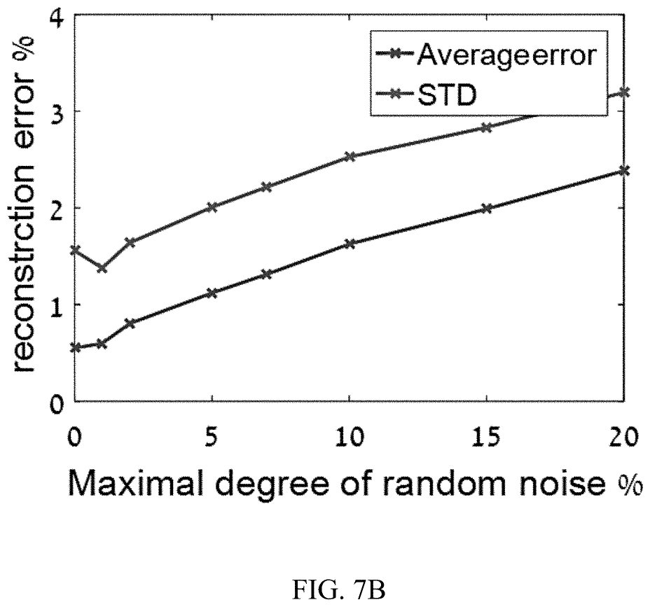

FIGS. 6A-6B show preliminary numerical results of chiral mixture reconstruction using the present reconstruction algorithms. FIG. 6A shows reconstruction statistics with various levels of assumed noise. FIG. 6B shows average reconstruction error and standard deviation of error vs. the assumed measurement noise. Ensemble of 1000 mixtures is used. Very small errors of <1% are obtained from the single-shot reconstruction, even if measurement noise is ˜5%.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Rather, the computer readable storage medium is a non-transient (i.e., not-volatile) medium.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The description of a numerical range should be considered to have specifically disclosed all the possible subranges as well as individual numerical values within that range. For example, description of a range from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system for determining chiral characteristic of an analyte, comprising:
at least one hardware processor; and
a non-transitory computer-readable storage medium having stored thereon program instructions, the program instructions executable by the at least one hardware processor to:
receive a target signal representing spectral emission associated with a target analyte comprising a multi-center chiral molecule; and
at an inference stage, apply a trained machine learning model to said target signal, to determine chiral characteristic of said target analyte.

2. The system of claim 1, wherein the trained machine learning model is produced by receiving a plurality of signals representing spectral emissions resulting from an interaction between a laser field and a respective plurality of analytes, wherein at least some of said analytes comprise multi-center chiral molecules, and
at a training stage, train said machine learning model on a training set comprising:
said plurality of signals, and
(ii) labels associated with a configuration of a chirality in each of said plurality of analytes, wherein said plurality of signals are labeled with said labels.

3. The system of claim 2, wherein the laser field is locally chiral at said interaction.

4. The system of claim 3, wherein said laser field maintains said local chirality within all of an interaction region with each of said plurality of analytes and said target analyte.

5. The system of claim 2, wherein said laser field exhibits one of the following symmetry properties: static reflection symmetry; dynamical reflection symmetry; dynamical inversion symmetry; dynamical improper rotational symmetry; and lack of inversion, reflection, and improper-rotation symmetry.

6. The system of claim 2, wherein said laser field is generated by illuminating at least two laser beams non-collinearly, wherein at least one of the following is controlled: (i) one or more of the wavelengths of the laser beams, and (ii) one or more of the polarizations of the laser beams.

7. The system of claim 2, wherein said laser field has different handedness in different sections of the interaction region.

8. The system of claim 2, wherein said spectral emission is a harmonic spectral emission resulting from a high harmonic generation process between said laser field and each of said plurality of analytes and said target analyte.

9. The system of claim 2, wherein said spectral emission is a harmonic spectral emission resulting from a low-order harmonic generation process between said laser field and each of said plurality of analytes and said target analyte.

10. A method of determining chiral characteristic of an analyte, comprising:
receiving, by a processor, a target signal representing spectral emission associated with a target analyte comprising a multi-center chiral molecule; and
at an inference stage, applying a trained machine learning (ML) model to said target signal, to determine chiral characteristic of said target analyte.

11. The method of claim 10, wherein said trained ML model is produced by receiving a plurality of signals representing spectral emissions resulting from an interaction between a laser field and a respective plurality of analytes, wherein at least some of said analytes comprise multi-center chiral molecules; and
at a training stage, training the ML model on a training set comprising:
(i) said plurality of signals, and
(ii) labels associated with a configuration of a chirality in each of said plurality of analytes. wherein said plurality of signals are labeled with said labels.

12. The method of claim 11, wherein the laser field is locally chiral at said interaction.

13. The method of claim 11, wherein said laser field maintains said local chirality within all of an interaction region with each of said plurality of analytes and said target analyte.

14. The method of claim 11, wherein said laser field exhibits one of the following symmetry properties: static reflection symmetry; dynamical reflection symmetry; dynamical inversion symmetry; dynamical improper rotational symmetry; and lack of inversion, reflection, and improper-rotation symmetry.

15. The method of claim 11, wherein said laser field is generated by illuminating at least two laser beams non-collinearly, wherein at least one of the following is controlled: (i) one or more of the wavelengths of the laser beams, and (ii) one or more of the polarizations of the laser beams.

16. The method of claim 11, wherein said laser field has different handedness in different sections of the interaction region.

17. The method of claim 11, wherein said spectral emission is a harmonic spectral emission resulting from a high harmonic generation process between said laser field and each of said plurality of analytes and said target analyte.

18. The method of claim 13, wherein said spectral emission is a harmonic spectral emission resulting from a low-order harmonic generation process between said laser field and each of said plurality of analytes and said target analyte.

19. A method comprising:
obtaining reference data comprising a plurality of reference signals representing spectral emissions resulting from an interaction between a laser field and a reference analyte comprising a chiral molecule, wherein molar concentrations of stereo-isomers in said analyte are known;
obtaining target data comprising a plurality of target signals representing spectral emissions resulting from an interaction between a laser field and a target analyte comprising said specified chiral molecule;
calculating reference phase data with respect to each of said reference signals;
deriving target phase data with respect to said target signals, by applying an optimization algorithm which minimizes an error between said target signals and said reference signals, based, at least in part, on said calculated reference phase data; and
reconstructing molar concentrations of stereo-isomers in said target analyte, based, at least in part, on said target phase data.

20. The method of claim 19, wherein said specified chiral molecules has n chiral centers, and wherein said reference data comprises at least 2ⁿ⁺¹−1 said signals.
